(19) United States
(12) Patent Application Publication
(10) Pub. No.: US 2022/0344044 A1
(43) Pub. Date: Oct. 27, 2022

(54) USER-CUSTOMIZED SKIN DIAGNOSIS SYSTEM AND METHOD

(71) Applicant: LULULAB INC., Seoul (KR)

(72) Inventors: Sang Wook YOO, Suwon-si, Gyeonggi-do (KR); Yongjoon CHOE, Seoul (KR)

(73) Assignee: LULULAB INC., Seoul (KR)

(21) Appl. No.: 17/617,010

(22) PCT Filed: Jun. 5, 2020
(86) PCT No.: PCT/KR2020/007317

§ 371 Date: Dec. 7, 2021

(30) Foreign Application Priority Data
Jun. 14, 2019 (KR) 10-2019-0070544

(51) Int. Cl. G16H 50/20; G06T 7/00; G06T 11/00; G16H 30/40; A61B 5/00

(52) U.S. Cl. CPC G16H 50/20; G06T 7/0012; G06T 11/00; G16H 30/40; A61B 5/0077; A61B 5/441; A61B 5/743; A61B 5/7267; G06T 2207/20081; G06T 2207/30201; G06T 2207/30088; G06T 2200/24; G06T 2210/41; A61B 2560/0431

(57) ABSTRACT

A user-customized skin diagnosis system includes: a portable skin condition measurement device for photographing a face of a user in a contactless manner; and a skin analysis device that receives an image of the user's face photographed from the portable skin condition measurement device, analyzes the received image for at least one skin analysis item based on preset image processing and deep learning, and generates and displays skin diagnosis information and customized recommendation information according to the analyzed result.

### TECHNICAL FIELD

The present invention relates to a system and a method of using a portable skin condition measurement device to perform skin diagnosis for each user and provide feedback on user-customized diagnosis results.

### BACKGROUND ART

As interest in beauty increases regardless of gender and age, interest in skin care also increases. Due to the above trend, skin condition measurement devices capable of photographing a user's face to analyze various skin troubles (such as wrinkles, pores and acne) have been developed and commercialized.

In the related art, as a skin condition measurement type, a type has been used in which the user directly pushed the face into the inside of a device to photograph the skin condition so as to apply a preset photographing environment (for example, photographing parameters such as light source intensity, direction, aperture value, or shutter speed).

In this regard, Korean Patent Registration No. 10-1738417 (entitled “DEVICE AND METHOD FOR DIAGNOSIS AND TREATMENT OF SKIN CONDITION”) discloses a device for diagnosis and treatment of skin conditions, which includes: a housing having an internal space in which a face of a patient is accommodated; a face fixing unit for fixing the face in the internal space; a photographing unit for photographing the face in front of the face; and a control unit for controlling movements of the face fixing unit to photograph the face under the same conditions upon photographing the face.

The conventional skin condition measurement type has the advantage that a photographing position of the user is fixed, however, there is an inconvenience that the user can measure the skin condition only in a specific place or position.

Meanwhile, many users spend a lot of time and money selecting skin-related products for skin care, but have difficulty in selecting the product actually most suitable for the users among the many brands and products around the world. In general, consumers select skin-related products suitable for them through online information such as the Internet. However, since the products selected through searches or recommendations are subject to subjective criteria of other users, the products may not be the perfect solution.

Accordingly, there is a need for a system that enables the user to easily measure a skin condition of the user anytime and anywhere, and feeds back an accurate skin diagnosis result to the user. Further, there is a need for a skin diagnosis system that can recommend information (e.g., product or solution) customized for each user's skin condition.

### DISCLOSURE

### Technical Problem

The embodiment of the present invention provides a system and a method of using a portable skin diagnosis device to easily measure a skin condition of a user, and feedback customized diagnosis results through skin measurement data analysis of the user.

In addition, the embodiment of the present invention provides a system and a method of using artificial intelligence (AI) technology to accurately analyze a skin condition of a user, and provide user-customized recommendation information as the analysis result.

However, the technical problems to be achieved by the present embodiments are not limited to the technical problems as described above, and other technical problems may exist.

### Technical Solution

As a technical solution for achieving the above-mentioned technical problem, the user-customized skin diagnosis system according to one aspect of the present invention includes a portable skin condition measurement device for photographing a face of a user in a contactless manner; and a skin analysis device that receives an image of the user's face photographed from the portable skin condition measurement device, analyzes the image for at least one skin analysis item based on preset image processing and deep learning, and generates and displays skin diagnosis information and customized recommendation information according to the analyzed result, wherein the skin diagnosis information includes scores for skin analysis items obtained by scoring a current skin condition of the user with respect to skin analysis items, and information on a care-required item including at least one item from a lowest score for the skin analysis items, and the customized recommendation information includes at least one piece of information corresponding to the information on the care-required item among related product information and related solution information previously stored in response to each of the skin analysis items.

The skin diagnosis information may further include an average score for all of the skin analysis items based on the scores for the skin analysis items, a current skin condition image displayed such that a region corresponding to the information on the care-required item among regions of the user's face in the image is identifiable, at least one of an improved skin condition image obtained by combining a virtual image for expressing an improved skin condition with a region corresponding to the care-required item among the regions of the user's face in the image and a deteriorated skin condition image obtained by combining a virtual image for expressing a deteriorated skin condition with the region corresponding to the care-required item among the regions of the user's face in the image, and at least one of simulation images in which frames are gradually changed between the current skin condition image and the at least one of the improved skin condition image and the deteriorated skin condition image.

In addition, the customized recommendation information may include at least one of, among the related product information and the related solution information corresponding to the information on the care-required item, information filtered based on user information including at least one of identification information, lifestyle information, gender, and age inputted in response to a skin diagnosis target user, information filtered based on living environment information including at least one of on weather, temperature, humidity, fine dust, and ultraviolet light, and information filtered based on related product review data and related solution data collected through natural language analysis processing on the web based on the skin diagnosis information.

In addition, the skin analysis device may include: a communication module for communicating data with the portable skin condition measurement device according to a preset communication format; a memory storing a skin analysis program; and a processor for executing the program stored in the memory, in which the processor may, based on execution of the skin analysis program, provide a user interface for enabling an input of the user information including the identification information, the lifestyle information, the gender and the age of the skin diagnosis target user, detect an entire face of the user by analyzing a plurality of face regions through the preset image processing upon reception of an image of the user's face through the communication module, digitize characteristic values of the skin analysis items for each part of the entire face of the user, and generate the scores for the skin analysis items based on the user information and the digitized characteristic values.

In addition, the skin analysis item may include at least one of pigmentation, redness, acne, pores, wrinkles and sebum.

In addition, the portable skin condition measurement device may include: a communication unit for communicating data with the skin analysis device according to a preset communication format; a photographing unit for photographing the user's face in a contactless manner; a lighting unit for outputting a light source for photographing a natural light image and a UV image during photographing operation of the photographing unit; and a control unit that controls operation of the lighting unit and operation of the photographing unit, and controls to transmit the image of the user's face photographed by the photographing unit through the communication unit.

According to another aspect of the invention, a user-customized skin diagnosis method through a user-customized skin diagnosis system including a portable skin condition measurement device and a skin analysis device include: receiving an image of a face of a user photographed in a contactless manner using the portable skin condition measurement device; analyzing the received image of the user's face according to at least one skin analysis item based on preset image processing and deep learning; and generating and displaying skin diagnosis information and customized recommendation information according to the analyzed result, wherein the skin diagnosis information includes scores for skin analysis items obtained by scoring a current skin condition of the user with respect to skin analysis items and information on a care-required item including at least one item from a lowest score for the skin analysis items, and the customized recommendation information includes and at least one piece of information corresponding to the information on the care-required item among related product information and related solution information previously stored in response to each of the skin analysis items.

In addition, the user-customized skin diagnosis system according to another aspect of the invention includes: a portable skin condition measurement device for photographing a face of a user in a contactless manner; a skin diagnosis server that receives an image of the user's face photographed from the portable skin condition measurement device, analyzes the image for at least one skin condition item based on deep learning, and provides skin diagnosis information and customized recommendation information according to the analyzed result; and a user terminal that receives the skin diagnosis information and the customized recommendation information provided from the skin diagnosis server, and displays the received customized recommendation information on a screen, wherein the user terminal is equipped with a dedicated application for processing information provided from the skin diagnosis server, the skin diagnosis information includes scores for skin analysis items obtained by scoring a current skin condition of the user with respect to skin analysis items, and information on a care-required item including at least one item from a lowest score for the skin analysis items, and the customized recommendation information includes and at least one piece of information corresponding to the information on the care-required item among related product information and related solution information previously stored in response to each of the skin analysis items.

### Advantageous Effects

According to the Technical Solution of the present invention described above, the user's skin can be accurately analyze by simply photographing the user face image in a non-contact manner, and the customized product and the solution information suitable for each user can be provided based on the analysis result.

According to the Technical Solution of the present invention described above, the user's skin can be accurately analyze by simply photographing the user face image in a non-contact manner, and the customized product and the solution information suitable for each user can be provided based on the analysis result.

In addition, according to the Technical Solution of the present invention, the user's individual skin age, the score for each skin analysis item according to the analysis result, the mainly concerned regions, and the like may be analyzed, so that the overall condition of the skin can be checked.

In addition, according to the Technical Solution of the present invention, the changes in the user skin based on time can be checked through data, so that data for observing skin improvement and aging can be provided.

In addition, according to the Technical Solution of the present invention, the skin changes before and after skin care may be simulated to enable the user to predict skin changes of the user, so that the need for care can be highlighted and the use of suitable products and the application of solutions can be guided.

### DESCRIPTION OF DRAWINGS

FIG. 1 is a schematic diagram of a user-customized skin diagnosis system according to one embodiment of the present invention.

FIG. 2 is a schematic diagram showing the portable skin condition measurement device and the skin diagnosis device according to one embodiment of the present invention.

FIG. 3 is a distribution diagram for explaining the numerical distribution calculation scheme upon calculating skin diagnosis results according to one embodiment of the present invention.

FIGS. 4 to 6 are examples of user interface screens on which user-customized skin diagnosis feedback results according to one embodiment of the present invention are outputted.

FIG. 7 is a flowchart for explaining the user-customized skin diagnosis method according to one embodiment of the present invention.

FIG. 8 is a schematic diagram of a user-customized skin diagnosis system according to another embodiment of the present invention.

### BEST MODE

### Mode for Invention

Hereinafter, embodiments of the present invention will be described in detail with reference to the accompanying drawings, so that a person having ordinary skill in the art may easily carry out the present invention. However, the invention may be embodied in various different forms and is not limited to the embodiments described herein. In addition, parts irrelevant to the description are omitted in the drawings to clearly describe the present invention, and like reference numerals designate like parts throughout the specification.

Throughout the specification, when a part “includes” a certain component, the above expression, unless particularly stated otherwise, does not exclude other elements but may further include the other elements, and does not preclude the possibility of the presence or addition of one or more other features, numbers, steps, elements, components, or combinations thereof.

In the present specification, the term ‘unit’ or ‘module’ includes a unit implemented by hardware, a unit implemented by software, and a unit implemented by both of the hardware and the software. In addition, one unit may be implemented using at least two pieces of hardware, and at least two units may be implemented by one piece of hardware. Meanwhile, “unit” may not be limited to software or hardware, may be configured to present in an addressable storage medium, and may be configured to reproduce at least one processor. Accordingly, as an example, the ‘unit’ includes components such as software components, object-oriented software components, class components, and task components, processes, functions, attributes, procedures, subroutines, segments of program code, drivers, firmware, microcode, circuits, data, databases, data structures, tables, arrays and variables. The functionality provided within the components and the ‘unit’s may be combined into a smaller number of components and ‘unit’s or further separated into additional components and the ‘unit’s. In addition, the components and ‘unit’s may be implemented to reproduce at least one CPU in a device or a secure multimedia card.

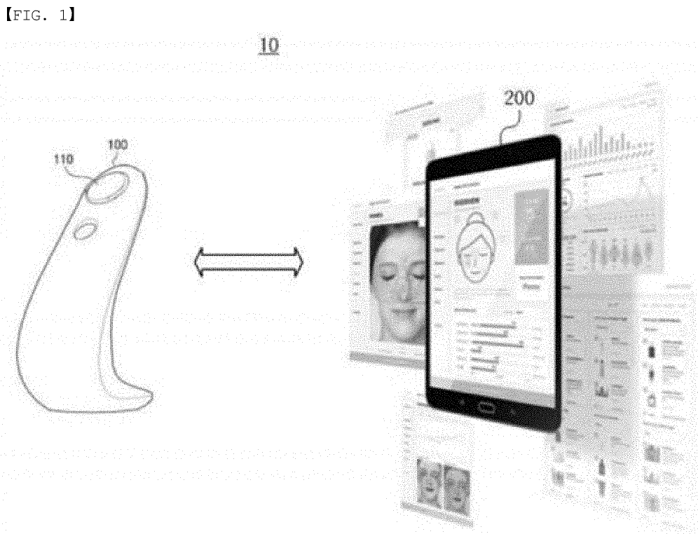

FIG. 1 is a schematic diagram of a user-customized skin diagnosis system according to one embodiment of the present invention.

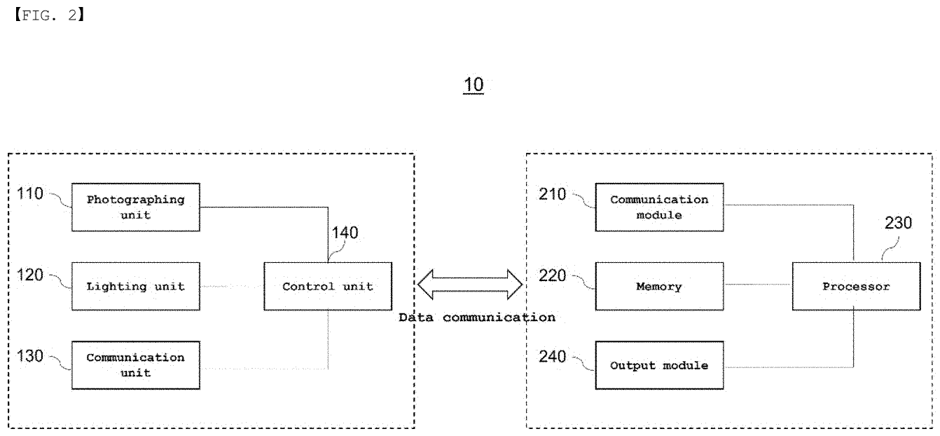

In addition, FIG. 2 is a schematic diagram showing the portable skin condition measurement device and the skin analysis device of FIG. 1.

As shown in FIG. 1, the user-customized skin diagnosis system 10 includes a portable skin condition measurement device 100 that can be carried by the user, and a skin analysis device 200 for performing data communication in conjunction with the portable skin condition measurement device 100.

The portable skin condition measurement device 100 photographs a face of the user in a contactless manner, and transmits the photographed image to the skin analysis device 200. The portable skin condition measurement device 100 accurately acquires the user's face image, and particularly measures the skin condition of the entire region of the face instead of the skin diagnosis result for a local region.

For the reference, according to one embodiment of the present invention, the term “portable” signifies that a device is movable to enable photographing at a position and a location desired by a user.

As shown in FIG. 2, the portable skin condition measurement device 100 includes: a photographing unit 110 for photographing a face of a user in a contactless manner; a lighting unit 120 for outputting a light source for photographing a natural light image and a UV image during photographing operation of the photographing unit 110; a communication unit 130 for communicating data with the skin analysis device 200 according to a preset communication format; and a control unit 140 controls the operation of the lighting unit 120 and the photographing unit 110, and controls to transmit the image of the user's face photographed through the photographing unit 110 to the outside through the communication unit 130.

The communication unit 130 may communicate with the skin analysis device 200 through wireless short-range communication, and may be implemented as, for example, a Bluetooth module, a Zigbee module, a Wi-Fi module, an NFC module, or the like. However, the communication scheme of the communication unit 130 is not limited thereto, and also may use wireless communication such as mobile communication or wired communication using a port or the like with the skin analysis device 200.

The lighting unit 120 may include at least one light source, and white light may be outputted by emitting, for example, at least one of red light, green light, and blue light. In order to distinguish from a UV user face image among user face images photographed by applying the light source outputted by the lighting unit 120, a natural light image will be referred to as a general user face image.

As described above, the portable skin condition measurement device 100 and the skin analysis device 200 according to one embodiment of the present invention have been described as being operated in conjunction with each other as separate configurations. However, according to another embodiment of the present invention, the portable skin condition measurement device 100 and the skin analysis device 200 may be integrally designed. In this case, the portable skin condition measurement device 100 may include at least some of components of the skin analysis device 200 described below. For example, the portable skin condition measurement device integrated with the skin analysis device according to another embodiment of the present invention may further include an output unit (not shown) capable of displaying skin diagnosis results, customized recommendation information and the like on a screen. In addition, the control unit 140 of the portable skin condition measurement device 100 may perform all operations processed by the processor of the skin analysis device 200 described below.

Returning to FIG. 2, the control unit 140 may obtain a user face image by photographing the user's face based on an arbitrary photographing parameter through the photographing unit 110.

For example, the photographed user face image may be a single user face image for forming a single user face image described later, or may be a plurality of user face images for forming an entire user face image.

The “single user face image” refers to a single image obtained by the photographing unit 110 to analyze a skin condition and formed by the processor 130. The control unit 140 may generate the user face image, which is photographed on a basis of any one photographing parameter, as the single user face image through the photographing unit 110.

The “entire user face image” refers to a user face image formed by extracting and combining specific face regions from each of a plurality of user face images obtained on a basis of a plurality of photographing parameters, and used for skin condition measurement. The control unit 140 may generate the entire user face image by obtaining and combining a plurality of user face images photographed on the basis of the different photographing parameters through the photographing unit 110.

The photographing parameter may include intensity of the light source, direction of the light source, shutter speed, aperture value, exposure value and the like through the lighting unit 120, and the present invention is not limited thereto. The photographing parameters may be set based on preliminarily photographed values with respect to preset measurement items (e.g., brightness) for each region (e.g., at least one region based on a central region) of the user's face in advance. In other words, a measurement value (e.g., brightness value) in a predetermined range may be measured for a first region (e.g., central region) of the user's face in the case of preliminarily photographing by applying a first preliminary photographing parameter, and a measurement value (e.g., brightness value) in a predetermined range may be measured for a second region (e.g., any one of outer peripheral regions) of the user's face in the case of preliminarily photographing by applying a second preliminary photographing parameter. In this case, the first and second preliminary photographing parameters may be used as references for setting photographing parameters for obtaining the corresponding measurement values afterward.

The control unit 140 may extract the first region from a first user face image obtained by a first photographing parameter, and extract the second region from a second user face image obtained by a second photographing parameter to combine the first and second regions, so that the entire user face image for measuring the skin condition may be formed.

The control unit 140 may determine difference values in the vicinity of a boundary between the first region and the second region, correct the difference values, and then combine the regions.

In addition, the control unit 140 may detect motion information through a motion sensor (not shown) or the like of the portable skin condition measurement device 100, and correct the second user face image according to the degree of motion detected between photographing of the first region and the photographing of the second region.

Meanwhile, in the case that at least some regions are deviated when the user face is detected from the user face image photographed through the photographing unit 110, the control unit 140 may output information that suggests the user to change a position of the face and a distance from the photographing unit 110. The control unit 140 may output information that suggests to change a photographing condition through an output unit (e.g., a display, an audio output apparatus, or the like; not shown) of the portable skin condition measurement device 100.

The control unit 140 may measure a distance between the user face and the photographing unit 110 through a distance sensor (not shown) of the portable skin condition measurement device 100, and suggest to move the user face position and change the distance when the distance therebetween is beyond a preset value or a part of the face is separated from the photographed user face image.

The degree of separation of the user face region may be determined based on a case in which detection of outline of the user face in the user face image fails or the outline is cut off at the boundary. The outline of the face may be acquired through the technology such as landmark detection or edge detection.

Meanwhile, the skin analysis device 200 receives an image of the user's face photographed from the portable skin condition measurement device, analyzes the received image for at least one skin analysis item based on deep learning, and generates and displays skin diagnosis information and customized recommendation information according to the analyzed result.

The skin diagnosis information may include scores for skin analysis items obtained by scoring a current skin condition of the user with respect to skin analysis items, and information on a care-required item including at least one item from a lowest score for the skin analysis items.

In addition, the customized recommendation information may include and at least one piece of information corresponding to the information on the care-required item among related product information and related solution information previously stored in response to each of the skin analysis items.

Specifically, as shown in FIG. 2, the skin analysis device 200 includes a communication module 210, a memory 220, a processor 230 and an output module 240.

The communication module 210 communicates data with the portable skin condition measurement device 100 according to a preset communication format. In other words, the communication module 210 receives the user face image obtained by photographing the user face from the portable skin condition measurement device 100 and transmits the received user face image to the processor 230.

The memory 220 stores a skin analysis program for performing skin analysis and customized information recommendation based on the user face image. The above memory 220 is a generic term for a non-volatile storage that maintains stored information even when power is not supplied, and a volatile storage device that requires power to maintain stored information.

The processor 230 executes the program stored in the memory 220, and processes the following operations according to the execution of the skin analysis program.

Specifically, the processor 230 provides a user interface that allows the input of user information including identification information, lifestyle information, gender, and age of the skin diagnosis target user. The user interface is a concept including a graphic user interface (GUI) displayed on a screen through the output module 240. In addition, skin diagnosis information and customized recommendation information according to a skin condition analysis result described below may also be provided through the user interface.

When the photographed user face image is received through the communication module 210, the processor 230 detects an entire face of the user by analyzing a plurality of face regions through the preset image processing, digitizes characteristic values of the skin analysis items for each part of the entire face of the user, and generates the scores for the skin analysis items based on the user information and the digitized characteristic values.

In other words, the processor 230 collects scoring data for calculating a score for each skin analysis item through the control unit 140 of the portable skin condition measurement device 100, receives results obtained by filtering inappropriate images according to preset conditions with respect to the collected scoring data, and performs scoring calculating a numerical distribution on the filtered image.

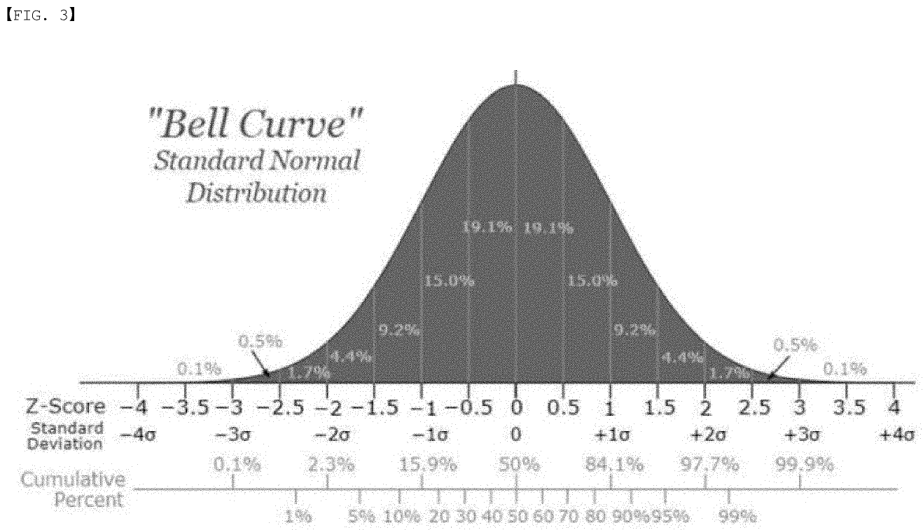

FIG. 3 is a distribution diagram for explaining the numerical distribution calculation scheme upon calculating skin diagnosis results according to one embodiment of the present invention.

The processor 230 may separately analyze previously collected images with respect to the preset skin analysis items such as wrinkle, melasma, and redness, respectively, and obtain one numerical value for each item.

For example, in the case of wrinkle item, the ratio of the wrinkle region is obtained numerically from the number of pixels detected as wrinkles compared to the number of pixels corresponding to the skin region in the collected image.

Based on these numerical values, the distribution of numerical values may be modeled according to gender, and age sections for each skin analysis item. For example, when assuming the gender has two cases such as male or female, the age has 10 sections by every 10 years, and there are six preset skin analysis items, a total of 120 numerical distributions are obtained. FIG. 3 shows a normal distribution as an example of the distribution that can be used upon modeling the above numerical distributions.

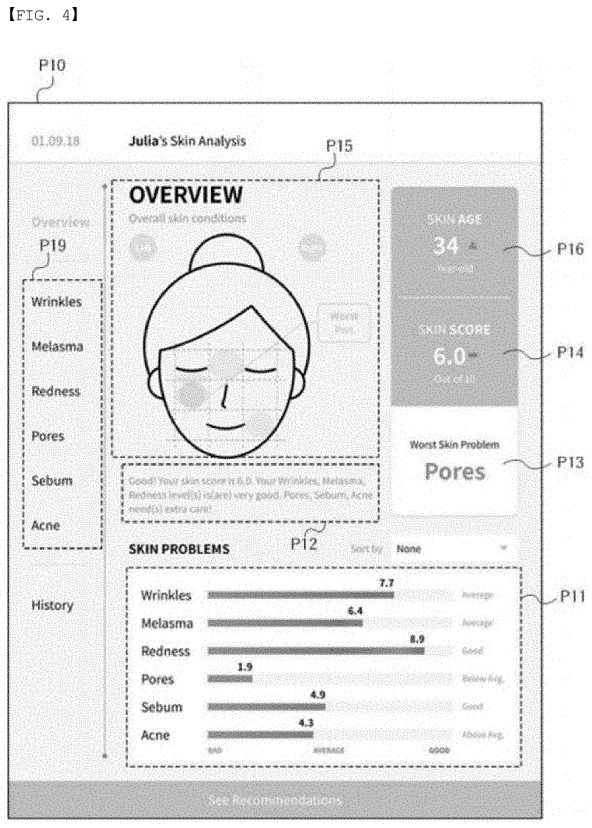

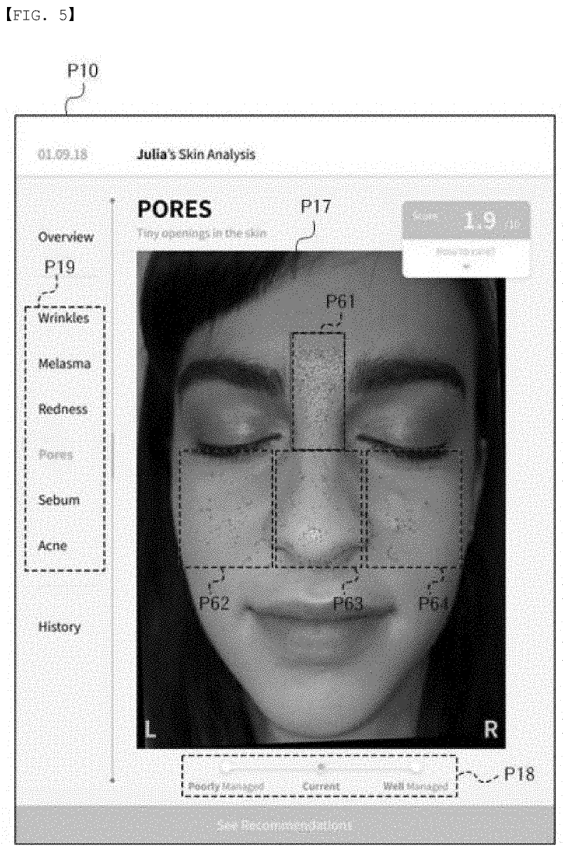

FIGS. 4a to 5 are examples of user interface screens on which user-customized skin diagnosis feedback results according to one embodiment of the present invention are outputted.

Referring to FIGS. 4a and 4b, the preset skin analysis item includes wrinkles, melasma, redness, pores, sebum and acne. However, the type of skin analysis item is not limited thereto.

The processor 230 may separate the received user face image (i.e., single user face image or entire user face image) into a melamine image and a hemoglobin image by applying the pigment separation technology, so that the melamine image may be analyzed to obtain information on melasma level and skin tone, and the hemoglobin image may be analyzed to obtain information on redness present on the user face.

In addition, the processor 230 may analyze RGB of the received user face image, thereby obtaining information on acne present on the user face (e.g., occurrence of acne, and probability of occurrence of acne), information on pores present on the user face (e.g., presence of pores, and depth of pores), and information on wrinkle present on the user face (e.g., existence of wrinkles, and depth of wrinkles).

In addition, the processor 230 may obtain information on sebum present on the user face from the received UV image of the user face image.

In the above step of digitizing the characteristic value for each skin analysis item by the processor 230, skin photographing data for a plurality of users may be analyzed based on deep learning.

The processor 230 may map scores within a preset range (e.g., 0 to 10) depending on a position where the corresponding numerical value exists on the distribution, according to the result of digitizing the characteristic value for the user face of each skin analysis item as shown in FIG. 3. For example, in FIG. 3, 0 point may be mapped when it is μ+2σ or more of the melasma area distribution, and 10 points may be mapped when it is μ−2σ or less of the melasma area distribution.

For example, the user may be notified of a care target region (that is, a skin region relatively necessary for more care) for each item through the user interface, based on the analysis results for the six items. The skin condition for each item may be scored and indicated by points between 0 and 10, and the overall skin condition may be scored and expressed by points between 0 to 10 based on the total analysis result, based on previously collected analytical values with respect to people having the same gender and similar age.

Referring back to FIG. 4, through the user interface P10, the processor 230 may provide, as skin diagnosis information, an each skin analysis item scoring graph P11 for scoring a current skin condition of the user, text information P12 for each skin analysis item score and a care-required item, information P13 on a care-required item including at least one item from a lowest score for the skin analysis items, an average score P14 for all of the skin analysis items based on the scores for the skin analysis items, a current skin condition image P15 displayed such that a region corresponding to the information on the care-required item among regions of the user's face in the image is identifiable, and a skin age P16.

In addition, when the overall analysis result for each skin analysis item is expressed in the current skin condition image P15, through the user interface P10, the processor 230 may further provide at least one of an improved skin condition image obtained by combining a virtual image for expressing an improved skin condition and a deteriorated skin condition image P17 obtained by combining a virtual image for expressing a deteriorated skin condition with a region corresponding to the care-required item among the regions of the user face in the image for each skin analysis item, as shown in FIG. 5.

FIG. 5 shows that “pores” are selected as the care-required skin analysis item, and shows that at least one care-required region P61 to P64 among the entire region of the user face is separately displayed to be identifiable for the corresponding skin analysis item. In addition, FIG. 4b shows a user interface including a toggle button P18 for outputting a simulation image having frames gradually changed between the current skin condition image, the improved skin condition image and the deteriorated skin condition image.

The improved skin condition image shown in FIG. 5 may be selectively outputted for each skin analysis item by using each item selection button P19 of FIG. 4a.

Accordingly, the image processing technology is used for a condition when the user's skin condition is improved/deteriorated, so that a face image upon the improved/deteriorate skin condition may be simulated and includes, for example, combining a face image when the degree of pigmentation is severe or improved, combining a face image when sizes of pores are larger or smaller, combining a face image when acne is cured or deteriorated, and combining a face image when wrinkles are deepened or improved and disappeared.

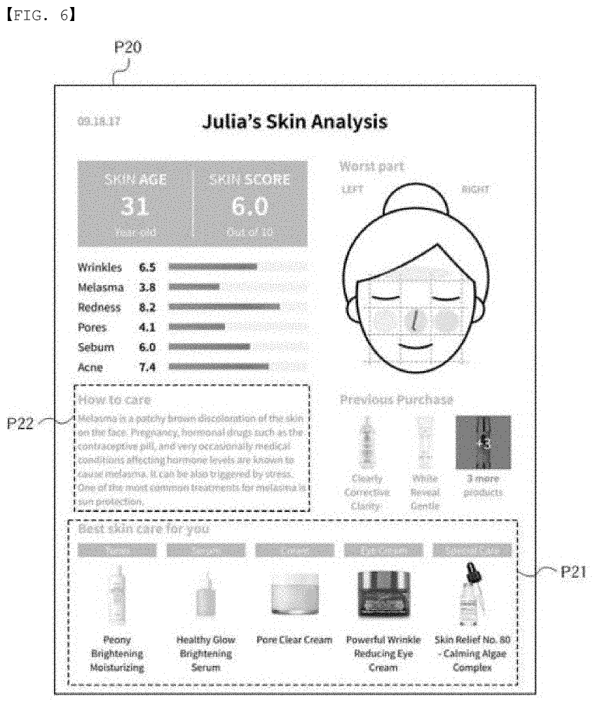

Referring to FIG. 6, the processor 230 may provide at least one piece of information P21 and P22 corresponding to the information on the analyzed care-required item among related product information and related solution information previously stored in response to each of the skin analysis items, as personalized recommendation information, through the user interface P20. In other words, based on the skin analysis results, a product for improving the worst skin item may be recommended from a previously collected product database, or a product preferred by other users having the same skin trouble may be retrieved and recommended to the user through the natural language analysis technology of product review data on the Internet.

The processor 230, among the related product information and the related solution information corresponding to the information on the care-required item, may output information filtered based on user information including at least one of identification information, lifestyle information, gender, and age inputted in response to a skin diagnosis target user.

For example, a product suitable for the age and gender of the user, a product reflecting the user's lifestyle information, and the like may be provided as recommended information.

Specifically, the processor 230, through the user interface P20, may provide personalized recommendation information includes at least one of information filtered based on living environment information including at least one of on weather, temperature, humidity, fine dust, and ultraviolet light, and information filtered based on related product review data and related solution data collected through natural language analysis processing on Internet based on the skin diagnosis information.

For example, the processor 230 may recommend ingredients related to moisture and skin barrier regeneration care to a user mainly lives indoors, and may recommend a product having sunscreen ingredients or whitening care ingredients to a user mainly active outdoors. In addition, for a user having a main concern on wrinkles, a moisturizing product may be recommended to the user in twenties, and a regenerative care products may be mainly recommended to the user in forties. In addition, on a day with severe fine dust, the processor 230 may apply weight to a product having high ingredient density in the cica series, and provide product or solution information to protect the skin barrier.

Meanwhile, the processor 230 may provide a process that allows the user to purchase the recommended product, through the user interface P20.

For example, through the user interface P20, the processor 230 may provide a link to an associated purchasing website for the recommended product to provide a process for enabling one-time purchase.

For another example, the processor 230 may provide a user interface for enabling the user to request regular delivery of the recommended product. When the user selects the regular delivery for the recommended product, the processor 230 may automatically send the regular delivery request to a server (not shown) of a previously contracted (or affiliated) product brand. The regular delivery request may include user information (e.g., name, contact information, address, and the like) registered by the user through the user interface in advance. In addition, the processor 230 may further handle a cost processing process that allows the user to pay for the regular delivery before the regular delivery request is sent.

For still another example, the processor 230 may provide a user interface so that the user can register a terminal (e.g., smart phone or the like) of the user, may transmit application information, which allows the user to directly request the regular delivery of the recommended product when the user terminal is registered through the user interface, to the corresponding user terminal. The processor 230 may transmit access information (e.g., a link) to a previously associated skin diagnosis service server (not shown) to the user terminal. The skin diagnosis service server provide data so that the application for allowing the user to request the regular delivery of the recommended product is downloaded to the connected user terminal.

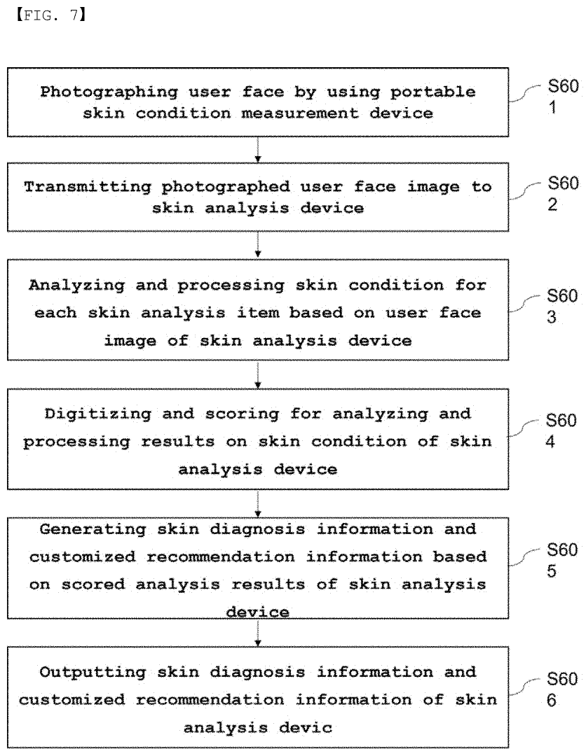

Next, a user-customized skin diagnosis service method according to one embodiment of the present invention will be described in detail with reference to FIG. 7.

FIG. 7 is a flowchart for explaining the user-customized skin diagnosis service method according to one embodiment of the present invention.

As shown in FIG. 7, first, the user face is photographed in a non-contact manner at a predetermined distance by using the portable skin condition measurement device 100 (S601).

The following steps may be performed through the processor 230 of the skin analysis device 200.

When the image (that is, the user face image) photographed in step S601 is received through the communication module (S602), the skin condition for each preset skin analysis item for the entire user face is analyzed based on deep learning on the received image (S603).

The skin analysis device 200 stores skin condition analysis results (that is, big data) previously accumulated for a predetermined period for each of a plurality of users, and the deep learning model is used to analyze a skin condition of each skin analysis item of the corresponding user.

For example, as described above, the skin analysis item may be set to an item including wrinkles, pigmentation, redness, acne, pores, and sebum.

As a result of the skin condition analysis, the current skin condition for each skin analysis item of the user is digitized and scored (S604).

Next, skin diagnosis information and customized recommendation information are generated based on the scored analysis result (S605), and the generated information is displayed on the screen (S606).

The skin diagnosis information may include the result of simulation based on deep learning technology by image-processing the conditions before and after the user skin change, and includes scores for skin analysis items obtained by scoring a current skin condition of the user with respect to skin analysis items, and ‘information on a care-required item’ including at least one item from a lowest score for the skin analysis items.

In addition, the customized recommendation information may include information that recommends products suitable for the user based on preset rules, and information that recommends products preferred or recommended by other users having similar skin conditions based on review data and the like of other users. The customized recommendation includes at least one piece of information corresponding to the information on the care-required item among related product information and related solution information previously stored in response to each of the skin analysis items.

Further, the user-customized skin diagnosis service method according to one embodiment of the present invention may further include a step of providing a user interface that allows the user to purchase the recommended products based on the results of the skin condition analysis of the user.

For example, a method of providing a link to purchase the recommended product at an associated purchasing site, a method of requesting a previously affiliated product brand server to regularly delivery the product to user, and a method of providing application data that allows the user to directly request the regular delivery of the product to the user terminal may be provided.

Meanwhile, according to another embodiment of the present invention, the user-customized skin diagnosis system may perform all the operations of the skin analysis device described above, and may also be implemented as a skin analysis server that communicates with at least one portable skin condition measurement device.

The “user terminal” referred to below may be implemented as a computer or portable terminal that can access a server or other terminals through a network. The computer includes, for example, notebook computers, desktops, laptops, and the like installed with a web browser. The portable terminal refers to a wireless communication device that guarantees portability and mobility, and may include, for example, all types of handheld-based wireless communication devices such as International Mobile Telecommunication (IMT)-2000, Code Division Multiple Access (CDMA)-2000, W-Code Division Multiple Access (W-CDMA), Long Term Evolution (LTE) communication-based terminals, smartphones, and tablet PCs.

In addition, the “network” may be implemented by all types of wired network such as a local area network (LAN), a wide area network (WAN) or a value added network (VAN), and wireless network such as a mobile radio communication network, or a satellite network.

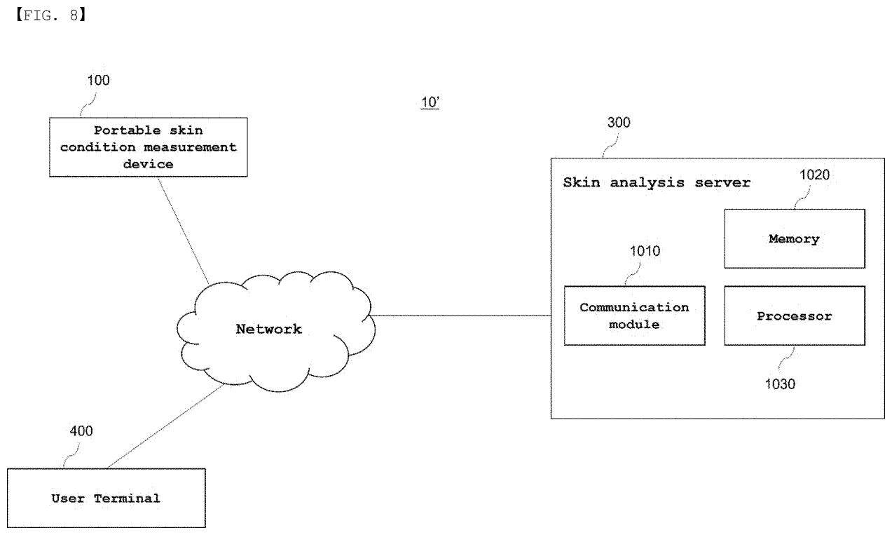

FIG. 8 is a schematic diagram of a user-customized skin diagnosis system according to another embodiment of the present invention.

As shown in FIG. 8, the user-customized skin diagnosis system 10′ according to another embodiment of the present invention includes a portable skin condition measurement device 100′, a skin analysis server 300 communicating with at least one portable skin condition measurement device 100′ to collect skin condition measurement information of a plurality of users, and a user's terminal (hereinafter referred to as “user terminal”) 400 that photographs a skin condition through the portable skin condition measurement device 100′.

The portable skin condition measurement device 100′ processes operations the same as or similar to the portable skin condition measurement device 100 previously described with reference to FIGS. 1 to 7. Accordingly, the description of configurations other than the different configuration of the portable skin condition measurement device 100′ according to another embodiment of the present invention will be omitted.

The portable skin condition measurement device 100′ accesses the skin analysis server 300 having a preset address through the network, and transmits the user face image photographed in a non-contact manner together with user information.

In addition, the skin analysis server 300 processes operations the same as or similar to the skin analysis device 200 previously described with reference to FIGS. 1 to 7. Accordingly, the description of configurations other than the different configuration of the skin analysis server 300 according to another embodiment of the present invention will be omitted.

The skin analysis server 300 includes a communication module 1010, a memory 1020, and a processor 1030, in which the processor 1030 analyzes a skin condition from the photographed user face image received from the portable skin condition measurement device 100′.

The processor 1030 provides skin analysis information and customized recommendation information as analysis results based on the received image to the user terminal 400 of the user connected to the skin analysis server 300.

To this end, the processor 1030 provides the user interface previously described with reference to FIGS. 4 to 6 to the user terminal 400 connected to the skin analysis server 300.

The user interface may be provided through a separate dedicated application, and the processor 1030 may provide the user terminal 400 to download corresponding dedicated application data.

Each configuration and method of the user-customized skin diagnosis system according to the embodiment of the present invention described above may be implemented in the form of a recording medium containing instructions executable by a computer, such as a program module executed by the computer. The computer readable medium may be any available media that is accessible by the computer, and include a volatile or non-volatile media, and a removable or non-removable media. In addition, the computer readable medium may include a computer storage medium. The computer storage medium includes all of volatile, non-volatile, removable and non-removable media implemented by any method or technology for storing information such as computer readable instructions, data structures, program modules or other data.

Although the method and system of the present invention have been described in connection with specific embodiments, some or all of components or operations thereof may be implemented using a computer system having a general hardware architecture.

The above description of the present invention is merely for illustration, it will be apparent that a person having ordinary skill in the art may carry out various deformations and modifications within the scope without departing from the idea of the present invention, the following claims and equivalents thereof. Therefore, the above described embodiments should be understood in all respects as illustrative and not restrictive. For example, each component described as unitary may be implemented in a distributed manner. Likewise, components that are described as distributed may also be implemented in a combined form.

The scope of the invention is indicated by the following claims rather than the above detailed descriptions, and all deformations or modifications derived from the idea and scope of the claims and their equivalents should be construed as being included in the scope of the present invention.

### DESCRIPTION OF REFERENCE NUMERALS

10, 10′: User-customized skin diagnosis and feedback system
100, 100′: Portable skin condition measurement device
200: Skin analysis device
300: Skin analysis server
400: User terminal

1. A user-customized skin diagnosis system comprising:
a portable skin condition measurement device for photographing a face of a user in a non-contact manner; and
a skin analysis device that receives an image of the user's face photographed from the portable skin condition measurement device, analyzes the image for at least one skin analysis item based on preset image processing and deep learning, and generates and displays skin diagnosis information and customized recommendation information according to the analyzed result, wherein
the skin diagnosis information includes scores for skin analysis items obtained by scoring a current skin condition of the user with respect to skin analysis items, and information on a care-required item including at least one item with a lowest score from among the skin analysis items, and
the customized recommendation information includes at least one piece of information corresponding to the information on the care-required item among related product information and related solution information previously stored in response to each of the skin analysis items.

2. The user-customized skin diagnosis system of claim 1, wherein the skin diagnosis information further includes an average score for all of the skin analysis items based on the scores for the skin analysis items, a current skin condition image displayed such that a region corresponding to the information on the care-required item among regions of the user's face in the image is identifiable, at least one of an improved skin condition image obtained by combining a virtual image for expressing an improved skin condition with a region corresponding to the care-required item among the regions of the user's face in the image and a deteriorated skin condition image obtained by combining a virtual image for expressing a deteriorated skin condition with the region corresponding to the care-required item among the regions of the user's face in the image, and at least one of simulation images where frames between the current skin condition image and the at least one of the improved skin condition image and the deteriorated skin condition image are gradually changed.

3. The user-customized skin diagnosis system of claim 1, wherein the customized recommendation information includes at least one of, among the related product information and the related solution information corresponding to the information on the care-required item, information filtered based on user information including at least one of identification information, lifestyle information, gender, and age inputted in response to a skin diagnosis target user, information filtered based on living environment information including at least one of on weather, temperature, humidity, fine dust, and ultraviolet light, and information filtered based on related product review data and related solution data collected through natural language analysis processing on Internet based on the skin diagnosis information.

4. The user-customized skin diagnosis system of claim 1, wherein the skin analysis device includes:
a communication module for communicating data with the portable skin condition measurement device according to a preset communication format;
a memory storing a skin analysis program; and
a processor for executing the program stored in the memory, wherein
the processor, based on execution of the skin analysis program, provides a user interface for enabling an input of the user information including the identification information, the lifestyle information, the gender and the age of the skin diagnosis target user, detects an entire face of the user by analyzing a plurality of face regions through the preset image processing upon reception of an image of the user's face through the communication module, digitizes characteristic values of the skin analysis items for each part of the entire face of the user, and generates the scores for the skin analysis items based on the user information and the digitized characteristic values.

5. The user-customized skin diagnosis system of claim 1, wherein the skin analysis item includes at least one of pigmentation, redness, acne, pore, wrinkle and sebum.

6. The user-customized skin diagnosis system of claim 1, wherein the portable skin condition measurement device includes:
a communication unit for communicating data with the skin analysis device according to a preset communication format;
a photographing unit for photographing the user's face in a contactless manner;
a lighting unit for outputting a light source for photographing a natural light image and a UV image during photographing operation of the photographing unit; and
a control unit that controls operation of the lighting unit and operation of the photographing unit, and controls to transmit the image of the user's face photographed by the photographing unit through the communication unit.

7. A user-customized skin diagnosis method through a user-customized skin diagnosis system including a portable skin condition measurement device and a skin analysis device, the user-customized skin diagnosis method comprising:
receiving an image of a face of a user photographed in a contactless manner using the portable skin condition measurement device;
analyzing the received image of the user's face according to at least one skin analysis item based on preset image processing and deep learning; and
generating and displaying skin diagnosis information and customized recommendation information according to the analyzed result, wherein
the skin diagnosis information includes scores for skin analysis items obtained by scoring a current skin condition of the user with respect to skin analysis items, and information on a care-required item including at least one item from a lowest score for the skin analysis items, and
the customized recommendation information includes at least one piece of information corresponding to the information on the care-required item among related product information and related solution information previously stored in response to each of the skin analysis items.

8. A user-customized skin diagnosis system comprising:
a portable skin condition measurement device for photographing a face of a user in a contactless manner;
a skin diagnosis server that receives an image of the user's face photographed from the portable skin condition measurement device, analyzes the image for at least one skin condition item based on deep learning, and provides skin diagnosis information and customized recommendation information according to the analyzed result; and
a user terminal that receives the skin diagnosis information and the customized recommendation information provided from the skin diagnosis server, and displays the received customized recommendation information on a screen, wherein
the user terminal is equipped with a dedicated application for processing information provided from the skin diagnosis server,
the skin diagnosis information includes scores for skin analysis items obtained by scoring a current skin condition of the user with respect to skin analysis items and information on a care-required item including at least one item from a lowest score for the skin analysis items, and
the customized recommendation information includes at least one piece of information corresponding to the information on the care-required item among related product information and related solution information previously stored in response to each of the skin analysis items.
